(19) United States
(12) Patent Application Publication
(10) Pub. No.: US 2024/0225705 A1
(43) Pub. Date: Jul. 11, 2024

(54) SYSTEMS AND METHODS FOR BONE FIXATION ANCHOR, PLATE, AND SPACER DEVICES

(71) Applicant: GLOBUS MEDICAL, INC., AUDUBON, PA (US)

(72) Inventors: Brian Flint, Fort Washington, PA (US); Christina Tiongson, Norristown, PA (US); James Gault, Philadelphia, PA (US)

(21) Appl. No.: 18/611,012

(22) Filed: Mar. 20, 2024

Related U.S. Application Data
(63) Continuation of application No. 17/477,742, filed on Sep. 17, 2021, now Pat. No. 11,957,389.
Application No. 17/477,742 is a continuation of application No. 16/122,995, filed on Sep. 6, 2018, now Pat. No. 11,147,599.
Application No. 16/122,995 is a continuation-in-part of application No. 15/238,772, filed on Aug. 17, 2016, now Pat. No. 10,687,873.

(51) Int. Cl. A61B 17/80; A61B 17/00; A61B 17/17; A61B 17/70; A61B 17/86

(52) U.S. Cl. CPC A61B 17/8042; A61B 17/1728; A61B 17/7059; A61B 17/8014; A61B 17/8052; A61B 17/8057; A61B 17/8061; A61B 17/86; A61B 17/8605; A61B 2017/00858; A61B 17/8047; A61B 17/808; A61B 17/809

(57) ABSTRACT

Devices, systems, and methods of bone fixation. The bone fixation system includes a bone plate having an upper surface and a lower surface configured to be in contact with bone, the bone plate having a locking hole extending from the upper surface to the lower surface. The locking hole is configured to receive a locking fastener. The locking fastener has a threaded head portion configured to engage and lock to the bone plate.

### CROSS-REFERENCE TO RELATED APPLICATIONS

This application is a continuation application of U.S. patent application Ser. No. 17/477,742 filed on Sep. 17, 2021 (published as U.S. Pat. Pub. No. 2022-0000526), which is a continuation application of U.S. patent application Ser. No. 16/122,995 filed on Sep. 6, 2018, now U.S. Pat. No. 11,147,599, which is a continuation-in-part of U.S. patent application Ser. No. 15/238,772, filed on Aug. 17, 2016, now U.S. Pat. No. 10,687,873, each of which are incorporated by reference herein in its entirety for all purposes.

### FIELD

The present invention relates to a bone fixation plate and fastener systems used to stabilize vertebrae and other bony anatomy. More specifically, the present invention relates to systems and methods for locking a fastener into a bone plate.

### BACKGROUND

Bones and bony structures are susceptible to a variety of weaknesses that can affect their ability to provide support and structure. Weaknesses in bony structures may have many causes, including degenerative diseases, tumors, fractures, and dislocations. Advances in medicine and engineering have provided doctors with a plurality of devices and techniques for alleviating or curing these weaknesses.

In the field of orthopedic surgery, and more specifically spinal surgery, bone fasteners may be used for fixation or for the fastening of orthopedic devices or instruments to bone tissue. An exemplary use of bone fastener may include using the bone fastener to fasten an orthopedic device, such as a bone plate, a spinal spacer, and/or a combination thereof, to a vertebral body for the treatment of a deformity or defect in a patient's spine. Bone fasteners can be secured to a number of vertebral bodies and a bone plate can then be connected to the vertebral bodies via the bone fasteners to fuse a segment of the spine. In another example, bone fasteners can be used to fix the location of a spinal spacer once the spacer is implanted between adjacent vertebral bodies. In yet another example, bone fasteners can be fastened to a number of vertebral bodies to anchor a spinal rod in place along a spinal column to treat a spinal deformity.

In the case of severely weakened bone, surgeons may face challenges in finding proper purchase of the bone fastener into the bone and proper attachment to the bone plate. Therefore, to overcome disadvantages noted above, the present disclosure provides bone fixation systems and methods using bone fasteners with threaded heads to engage and deform a textured portion of a bone plate.

### SUMMARY

To meet this and other needs, the present disclosure provides a bone fixation system having a bone plate and a locking fastener. The bone plate may include an upper surface and a lower surface that may be in contact with a bone. The bone plate may also include a locking hole extending from the upper surface to the lower surface, the locking hole may include a textured portion. The textured portion may include a texture that is a non-threaded surface. The locking fastener may include a head portion and a shaft portion and the locking fastener may be received by the locking hole and may be inserted into the bone. The head portion may be threaded and configured to engage the textured area of the locking hole.

The present disclosure also provides a bone fixation system including a spacer that may be inserted in between two adjacent vertebral bodies and a bone plate that may engage the spacer. The bone plate may have an upper surface and a lower surface that may be in contact with bone. The bone plate may further have a locking hole extending from the upper surface to the lower surface, the locking hole may include a textured portion and non-textured portion. The textured portion may include a texture that is a non-threaded surface. The bone fixation system may include a locking fastener that may be received in the locking hole and that may be inserted into the bone. The locking fastener may have a threaded head portion configured to lock to the bone plate. The threaded head portion may deform the textured portion of locking hole.

### BRIEF DESCRIPTION OF THE DRAWINGS

A more complete understanding of the present disclosure, and the attendant advantages and features thereof, will be more readily understood by reference to the following detailed description when considered in conjunction with the accompanying drawings wherein:

FIG. 1 illustrates an exemplary bone plate fixation device to used according to the principles of the present disclosure.

FIGS. 2 and 3 illustrate exemplary locking fasteners consistent with the principles of the present disclosure.

FIGS. 4 and 5 illustrate an exemplary bone plate consistent with the principles of the present disclosure.

FIGS. 6 and 7 illustrate exemplary bone plates and locking fasteners consistent with the principles of the present disclosure.

FIGS. 8-10 illustrates exemplary locking mechanisms consistent with the principles of the present disclosure.

### DETAILED DESCRIPTION

Embodiments of the disclosure are generally directed to devices, systems, and methods for bone stabilization. Specifically, embodiments are directed to bone plating with locking and/or non-locking fasteners for engaging with a bone fastener. The hole designs may allow for fixed angle and/or variable angle fixation. Systems and methods disclosed herein may allow for locking bone screws into a spinal plate or integrated plate-spacer to create a rigid construct and prevent back-out of screws while maintaining a preferred screw trajectory using a spherical or conical screw head profile and with tapering dual-lead threads at a specified torque. Some embodiments further include locking fasteners with self-forming threads configured to displace the plate material, thereby locking the fastener to the plate.

The present disclosure relates to exemplary embodiments of locking screws into a plate or integrated plate spacer. This may be accomplished through interference/cross-threading of tapered dual-lead threads on a head of the locking screw engaging with a screw hole or socket with a series of helical sweeps, diamond knurls, or similar relief cuts arranged about the central axis of the screw hole or socket. Tightening of the dual-lead threads of the screw head into these relief cuts creates rigid fixation of the screw and prevents loosening and screw back-out. As an example, the material of the screw may be the same as the plate/integrated-plate spacer or a harder material to promote controlled deformation and rigid fixation. The helical sweeps, diamond knurls, or similar relief cuts may allow for both fixed and variable angle locking screws with conical variability up to 10 degrees.

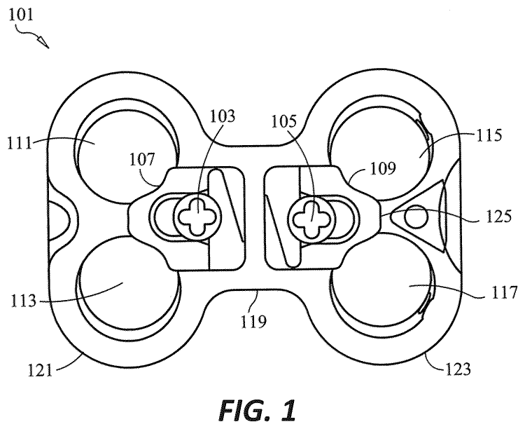

The plate(s) and/or plate-spacer device(s) may be adapted to contact one or more vertebral bodies. The configuration of the locking screw and/or screw hole of the present disclosure may be used by various plates and plate-spacer devices known in the art. Such exemplary bone plates and plate-spacer devices have been described, for example, in U.S. Pat. Nos. 9,326,802; 9,095,387; 9,044,275; and 9,044,275 which are incorporated herein by reference in their entireties. For purposes of illustration, one exemplary bone fixation plate is described in FIG. 1.

FIG. 1 shows one exemplary embodiment of a bone fixation plate 101 that may be used with the locking screw/screw hole configuration of the present disclosure. The plate may be secured to two vertebrae in order to maintain the vertebrae integrally with respect to one another in a desired orientation and at a desired spacing from one another. Plate 101 may include two fastening devices, such as screws 103-105 or the like, which are operatively communicable with plates 107-109. The plate also includes four openings 111-117, through which screws (not shown) may be used to fasten the plate 101 to the vertebrae.

Plate 101 and the screws may be comprised of any material, such as a metal, alloy, or any combination of the two. Preferably, the material used to construct the plate and the screws allows the plate 101 to maintain its structural integrity while allowing for a desired amount of resiliency. Furthermore, the material used is preferably bio-compatible and capable of withstanding the conditions of a body over a desired period of time. In some embodiments, this is achieved by manufacturing the plate 101 and screws using metals such as titanium or stainless steel. Titanium has sufficient ductility to permit a desired amount of curving of the plate 101 to conform to the shape of the vertebrae, yet has the strength to maintain its structural integrity.

In the exemplary embodiment of FIG. 1, bone fixation plate 101 comprises a center portion 119 and two distal portions 121-123. Each distal portion 121-123 may be attached to a different vertebra using fasteners, such as screws, that pass through openings 111-117.

Bone plates may be comprised of titanium, stainless steel, cobalt chrome, carbon composite, plastic or polymer—such as polyetheretherketone (PEEK), polyethylene, ultra-high molecular weight polyethylene (UHMWPE), resorbable polylactic acid (PLA), polyglycolic acid (PGA), combinations or alloys of such materials or any other appropriate material that has sufficient strength to be secured to and hold bone, while also having sufficient biocompatibility to be implanted into a body. Similarly, the fasteners may be comprised of titanium, cobalt chrome, cobalt-chrome-molybdenum, stainless steel, tungsten carbide, combinations or alloys of such materials or other appropriate biocompatible materials. Although the above list of materials includes many typical materials out of which bone plates and fasteners are made, it should be understood that bone plates and fasteners comprised of any appropriate material are contemplated.

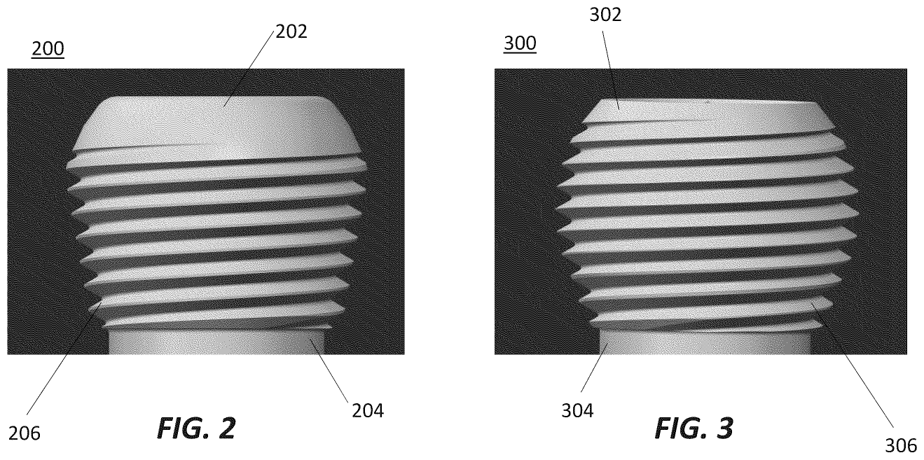

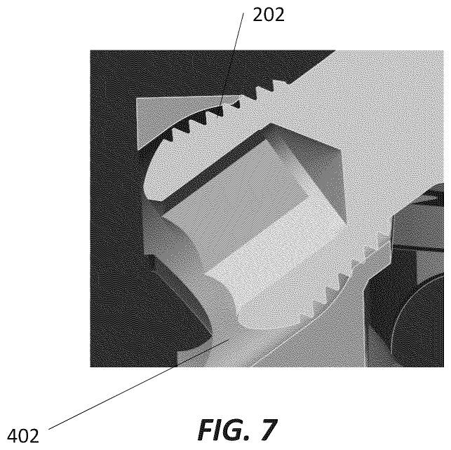

Referring now to FIGS. 2-10, exemplary embodiments of the present disclosure are presented. With regards to FIGS. 2-6, one or more locking fasteners may be inserted into a plate or integrated plate spacer through interference/cross-threading of tapered dual-lead threads on the screw head with a screw hole or socket with a series of helical sweeps, diamond knurls, or similar relief cuts arranged about the central axis of the screw hole or socket. Tightening of the dual-lead threads of the screw head into these relief cuts creates rigid fixation of the screw and prevents loosening and screw back-out.

The material of the screw may be the same as the plate/integrated-plate spacer or a harder material to promote controlled deformation and rigid fixation. The helical sweeps, diamond knurls, or similar relief cuts allow for both fixed and variable angle locking screws with conical variability up to 10 degrees.

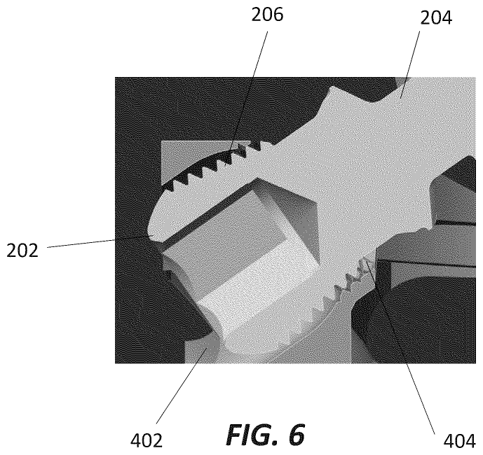

Specifically, locking fastener 200 of FIG. 2 may include a head portion 202 and a shaft portion 204 (shown in FIG. 6) configured to engage bone. Locking fastener 200 has a conical screw head profile with a textured area 206 with dual-lead threads. FIG. 3 illustrates a locking fastener 300 that has a screw head 302 and textured area 306. Screw head 302 has a spherical head profile with dual-lead threads. Each of bone screws 200 and 300 may be used under the principles of this disclosure according to any specific needs of a medical procedure and the preferences of a surgeon performing the medical procedure.

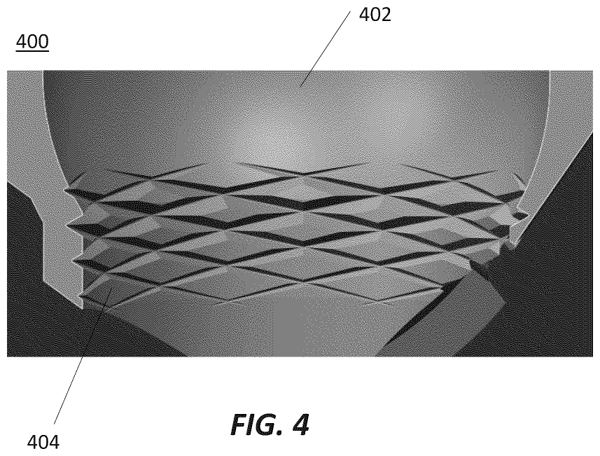

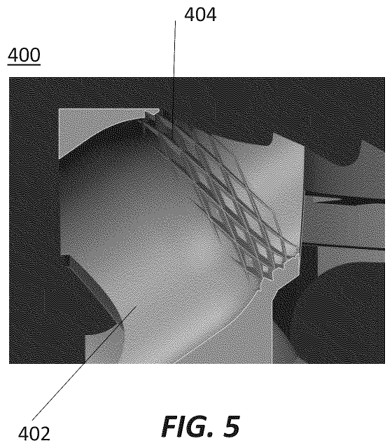

FIGS. 4 and 5 depict a plate 400 with a locking hole 402. An inside surface of screw hole 402 may be configured to have a textured portion 404, which may be, but not limited to, a series of helical sweeps, diamond knurls, threads, ridges, bumps, dimples, serrations or similar relief cuts arranged about the central axis of the screw hole or socket. The textured portion 404 may be of the same type (e.g., mating surfaces) or different from the textured area 206 of locking fastener 200. As shown, the textured portion 404 is disposed along an inner portion of the hole 402. The knurled surface may include straight, angled, or crossed lines cut or rolled into the material. For example, the textured portion 404 may take up about half or less of the surface area of hole 402.

An upper portion of the hole 402 may be tapered, without texturing, for example, to facilitate alignment of the fastener 200 with an opening of the locking hole 402. Locking hole 402 may be configured to receive a fixed or variable angle fastener 200. Locking hole 402 may be generally conical in shape such that it is wider near the top surface of plate 400 and narrower toward a bottom surface plate 400. The tapered portion and/or the textured portion 404 may be conical in shape.

In operation, shaft portion 204 may be threaded such that the fastener 200 may be threaded into the bone. The head portion 202 of the locking fastener 200 may include the textured area 206 around its outer surface that is sized and configured to engage with locking hole 402 of plate 400. Textured area 206 may include threads, ridges, bumps, dimples, serrations, or other types of textured areas. As shown, texture area 206 preferably includes a threaded portion extending substantially from the top of the head portion 202 to the bottom of the head portion 202 proximate to the shaft portion 204. The textured portion 404 of locking holes 402 may deform as head 202 interferes with the textured portion 404 of the hole 402, thereby providing a positive lock between the fastener 200 and the plate 400. Thus, as shown in FIG. 6, when locking fastener 200 is engaged with plate 400, textured area 206 is screwed into textured area 404 thereby deforming textured area 404 to lock fastener 200 into plate 400.

In an alternate embodiment, locking hole 402 may be configured to have a substantially smooth surface rather than having textured surface 404. In this embodiment, when locking fastener 200 is inserted into plate 400, textured area 206 digs into the substantially smooth inner surface of the locking hole 402 thereby locking fastener 200 into plate 400.

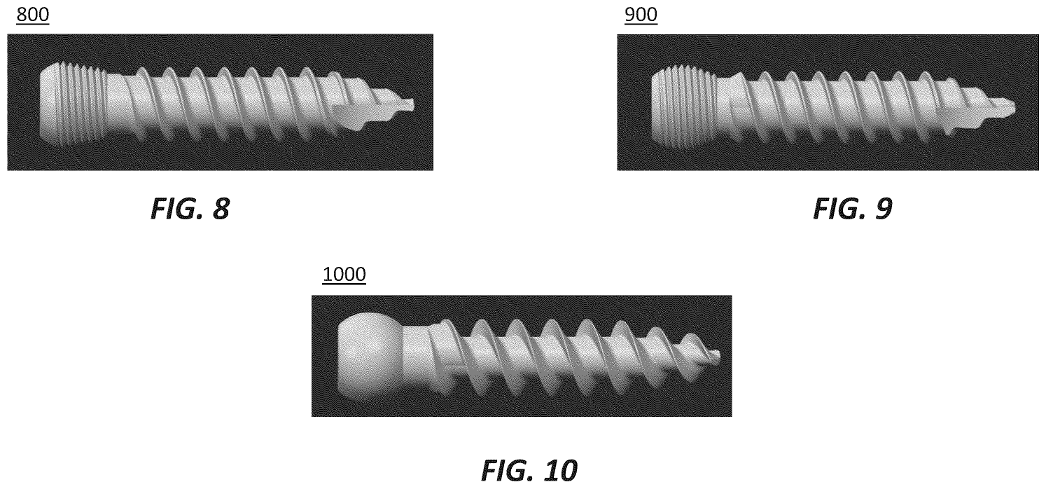

FIGS. 8-10 depict embodiments of bone screws 800, 900, and 1000 that may be used according to the principles of the present disclosure. Bone screws 800, 900, and 1000 maybe a fixed or variable angle 5.5 mm outside diameter (O.D.) screw. The screws may be spherically or conically tapered dual lead threads intended to create a rigid connection with a smooth screw hole or screw hole modified with helical sweeps, diamond knurls, or similar relief cuts. The tapering dual-lead threads may be configured to lock into a plate or integrated plate-spacer to provide a rigid stable construct in weak or osteoporotic bone. Additionally, the screw minor diameter and pitch may be modified to improve purchase in weak bone. The screw material may be TAV or cobalt-chromium (CoCr). A blocking screw may also be used to promote screw retention and further minimize the chance of back-out.

FIG. 8 depicts a fastener 800 that is a spherical head with a conical taper of the dual leads. FIG. 9 depicts a fastener 900 that is a spherical head with spherical thread taper variant. FIG. 10 depicts a non-locking, non-self-drilling variant with smaller minor diameter than that of fasteners 800 and 900.

The locking screw feature described above combined with large cancellous threads of the screw may allow for a rigid connection of the screw to the implant in cases where weakened bone prevents lagging of bone onto the implant surface. The screw-implant construct provides greater stability in patients with poor bone quality.

The cutting/wedging behavior of the conical threads allows for the use of a locking-type screw in the same socket or screw hole geometry as a non-locking lagging screw that uses a blocking screw feature for retention. This offers greater versatility the surgeon in the types of screws they can used for fixation depending on patient anatomy and bone quality while not altering existing lag screws.

Although the invention has been described in detail and with reference to specific embodiments, it will be apparent to one skilled in the art that various changes and modifications can be made without departing from the spirit and scope of the invention. Thus, it is intended that the invention covers the modifications and variations of this invention provided they come within the scope of the appended claims and their equivalents. It is expressly intended, for example, that all ranges broadly recited in this document include within their scope all narrower ranges which fall within the broader ranges. It is also intended that the components of the various devices disclosed above may be combined or modified in any suitable configuration.

What is claimed is:

1. A bone fixation system comprising:
a bone plate having an upper surface and a lower surface configured to be in contact with a bone, the bone plate having a textured portion; and
a locking fastener, having a head portion and a shaft portion, configured to configured to engage the textured portion and to be inserted into the bone.

2. The bone fixation system of claim 1, the bone plate having a locking hole that includes the textured portion and wherein the locking hole is generally conical in shape such that it is wider near the upper surface of the plate and narrower toward the lower surface of the plate.

3. The bone fixation system of claim 1, wherein the textured portion comprises a knurled surface.

4. The bone fixation system of claim 1, wherein the textured portion comprises diamond knurls.

5. The bone fixation system of claim 1, wherein the shaft portion comprises dual-lead threads for engagement with the bone.

6. The bone fixation system of claim 1, wherein the textured portion comprises a knurled surface including a pattern of straight, angled, or crossed lines cut into the plate.

7. The bone fixation system of claim 1, wherein the head portion is made of material that is harder than the textured portion.

8. The bone fixation system of claim 1, wherein the head portion is conical.

9. The bone fixation system of claim 1, wherein the head portion is spherical.

10. The bone fixation system of claim 1, further comprising a blocking screw configured to cover a portion of the locking fastener in a locked position to prevent the fastener from backing out of the plate.

11. A bone fixation system comprising:
a spacer configured to be inserted in between two adjacent vertebral bodies;
a bone plate having an upper surface and a lower surface configured to be in contact with a bone, the bone plate having a textured portion; and
a locking fastener, having a head portion and a shaft portion, configured to configured to engage the textured portion and to be inserted into the bone.

12. The bone fixation system of claim 11, the bone plate having a locking hole that includes the textured portion and wherein the locking hole is generally conical in shape such that it is wider near the upper surface of the plate and narrower toward the lower surface of the plate.

13. The bone fixation system of claim 11, wherein the textured portion comprises a knurled surface arranged about a central axis of the locking hole.

14. The bone fixation system of claim 11, wherein the textured portion comprises diamond knurls arranged about a central axis of the locking hole.

15. The bone fixation system of claim 11, wherein the shaft comprises dual-lead threads for engagement with the bone.

16. The bone fixation system of claim 11, wherein the textured portion comprises a knurled surface including a pattern of straight, angled, or crossed lines cut into the plate.

17. The bone fixation system of claim 11, wherein the threaded head portion is made of material that is harder than the textured portion.

18. The bone fixation system of claim 11, wherein the head portion is conical.

19. The bone fixation system of claim 11, wherein the head portion is spherical.

20. The bone fixation system of claim 11, further comprising a blocking screw configured to cover a portion of the locking fastener in a locked position to prevent the fastener from backing out of the plate.
